(19) United States
(12) Patent Application Publication
(10) Pub. No.: US 2018/0064742 A1
(43) Pub. Date: Mar. 8, 2018

(54) PET IMAGING TRACER FOR IMAGING PROSTATE CANCER

(71) Applicants: David WILSON, San Francisco, CA (US); THE REGENTS OF THE UNIVERSITY OF CALIFORNIA, Oakland, CA (US)

(72) Inventors: David WILSON, San Francisco, CA (US); Robert FLAVELL, San Francisco, CA (US)

(21) Appl. No.: 15/565,355

(22) PCT Filed: Apr. 11, 2016
(86) PCT No.: PCT/US2016/026980

§ 371 Date: Oct. 9, 2017

Related U.S. Application Data
(60) Provisional application No. 62/146,138, filed on Apr. 10, 2015.

(51) Int. Cl. A61K 31/7008; A61K 51/04; C07B 59/00

(52) U.S. Cl. CPC A61K 31/7008; A61K 51/0402; C07H 15/203; C07B 59/005; A61K 51/0491

(57) ABSTRACT

In various embodiments, the invention provides a radiotracer comprising a positron emitting atom bound to a deoxy sugar moiety. The radiotracer is reversible bound to a caging moiety that prevents or retards tissue uptake of the radiotracer while the caging moiety is in place. An exemplary caging moiety is acid labile and is cleaved upon uptake of the radiotracer by tissue with an acidic pH.

### CROSS REFERENCE TO RELATED APPLICATIONS

This application claims the benefit under 35 U.S.C. § 119(e) of U.S. Provisional Application No. 62/146,138 filed on Apr. 10, 2015, which is incorporated herein by reference in its entirety for all purposes.

### STATEMENT OF GOVERNMENT RIGHTS

This invention was made with Government support under Grant No. 5T32EB0011631-10 awarded by the National Institutes of Health. The Government has certain rights in the invention.

### FIELD OF THE INVENTION

The present invention relates generally to radioisotope labeled chemical precursors of ¹⁸F-fluorodeoxyglucose (FDG), designed to image the acidic tumoral microenvironment, and imaging methods using the radioisotope labeled compounds.

### BACKGROUND OF THE INVENTION

Prostate cancer is common and presents with variable clinical course, with an estimated 2.7 M men living with the disease in the USA. Prostate cancer (PCa) is the second leading cause of cancer-related death in men (Jemal, et al., CA Cancer J. Clin., 53:5-26 (2003)). Only one half of tumors due to PCa are clinically localized at diagnosis and one half of those represent extracapsular spread. Localization of that spread as well as determination of the total body burden of PCa have important implications for therapy, particularly as new combination and focal therapies become available. Also critically needed are targeted agents that can provide a readout on the biology of the tumor, with the ability to predict which tumors will lie dormant and which will develop into aggressive, metastatic disease. The current clinical standard for localizing cancer—including PCa—is shifting from the anatomic techniques such as computed tomography (CT) and magnetic resonance (MR) imaging to more physiologically relevant methods that employ molecular imaging, such as MR spectroscopy, single photon emission computed tomography (SPECT) and positron emission tomography (PET) (Geus-Oei, et al., Cancer Imaging, 8:70-80 (2008)). Such newer methods that utilize molecular imaging may provide the biological readout necessary for understanding tumor physiology, enabling more accurate prognosis and therapeutic monitoring. Molecular imaging may provide a way to not only detect tumors in vivo, but also to provide information regarding the biology of the lesion, if a mechanism-specific agent is used. For example, [¹⁸F]FDHT can be used to study the androgen receptor status of tumors (Larson, et al., The Journal of Nuclear Medicine, 45:366-373 (2004)).

Unlike many other cancers, PCa is particularly difficult to detect using existing molecular imaging tracers. There are several reasons for this, including the relatively slow growth and metabolic rate of PCa compared to other malignancies as well as the small size of the organ and proximity to the urinary bladder, into which most radiopharmaceuticals are eventually excreted.

Because of the relatively low metabolism of PCa, PET with [¹⁸F]fluorodeoxyglucose (FDG-PET) has proved ineffectual for diagnostic imaging of this disease. Other promising, experimental radiopharmaceuticals for imaging PCa are emerging, including those of the choline series (Scher, et al., European Journal of Nuclear Medicine Molecular Imaging, 34:45-53 (2007); Reske, et al., J. Nucl. Med., 47:1249-1254 (2006); Vees, et al., BJU International, 99:1415-1420 (2007)), radiolabeled acetates (Ponde, et al., The Journal of Nuclear Medicine, 48:420-428 (2007)), anti-1-amino-3-[¹⁸F]fluorocyclobutyl-1-carboxylic acid (anti[¹⁸F]F-FACBC) (Schuster, et al., The Journal of Nuclear Medicine, 48:56-63 (2007); Oka, et al., The Journal of Nuclear Medicine, 48:46-55 (2007)) 1-(2-deoxy-2-[¹⁸F]fluoro-L-arabinofuranosyl)-5-methyluracil ([¹⁸F]FMAU) (Tehrani, et al., The Journal of Nuclear Medicine, 48:1436-1441 (2007)) and [¹⁸F]fluorodihydrotestosterone ([¹⁸F]FDHT) (Larson, et al., The Journal of Nuclear Medicine, 45:366-373 (2004)). Each has its benefits and detriments, with no single agent ideal, i.e., easy to synthesize, little metabolism and demonstrating tumor-specific uptake, in all PCa phenotypes.

Overexpressed on most solid tumor neovasculature (Chang, et al., Cancer Research, 59:3192-3198 (1999)) as well as in prostate cancer, the prostate-specific membrane antigen (PSMA) is becoming an attractive target for cancer imaging and therapy (Zhou, et al., Nature Reviews/Drug Discovery, 4:1015-1026 (2005); Chang, Reviews in Urology, 6 (Suppl. 10):S13-S18 (2004)). PSMA-based agents can report on the presence of this marker, which is increasingly recognized as an important prognostic determinate in PCa (Murphy, et al., Urology, 51:89-97 (1998)). It is also the target for a variety of new PCa therapies (Galsky, et al., Journal of Clinical Oncology, 26:2147-2154 (2008)). ProstaScint™ is an ¹¹¹In-labled monoclonal antibody against PSMA that is clinically available for imaging PCa. ProstaScint™ and radiolabeled variations of this antibody are fraught with long circulation times and poor target to nontarget tissue contrast, limiting the utility of these agents (Lange, Urology, 57:402-406 (2001); Haseman, et al., Cancer Biother Radiopharm, 15:131-140 (2001); Rosenthal, et al., Techniques in Urology, 7(1):27-37 (2001)).

Owing to a low background and good spatial resolution positron emission tomography (PET) is a clinically relevant in vivo imaging modality. In fact, Mach and coworkers recently reported a PET radiotracer for detection of superoxide based on attachment of radioactive ¹⁸F onto the well-known dihydroethidium scaffold (Org. Biomol. Chem. 2014, 12, 4421). Other notable examples include [¹⁸F]fluorodeoxyglucose ([¹⁸F]FDG) and 3′-deoxy-3′-[¹⁸F]fluorothymidine ([¹⁸F]FLT), which are uptaken into rapidly proliferating cells and subsequently phosphorylated resulting in intracellular trapping of the radiotracer in these cells.

A combination of imaging tests are used for restaging patients with suspected or known metastatic prostate cancer. This results in high costs and high radiation burden for patients. FIG. 1. Thus, there is an unmet clinical need for a single imaging test for restaging prostate cancer. The present solves this need by providing novel tracers with improved signal to noise ratio for PET diagnosis, staging and restaging of cancer. Importantly, while the initial application of these tracers is in prostate cancer, this strategy is generally applicable to other cancers, and also to other pathologies including ischemia and infection.

### SUMMARY OF THE INVENTION

The present invention satisfies the long standing and unmet need for new tissue-specific compounds for imaging prostate cancer and angiogenesis. The present invention, in particular, provides imaging agents which differ from the prior art in modifications which were not previously known or suggested. Furthermore, the invention provides imaging agents that offer better contrast between target tissues and non-target tissues. The principle underlying the imaging agents' efficacy is equally applicable to agents for treatment of disease, e.g., cancer.

The present invention provides a new approach to reaction-based PET radiotracers. Uptake of exemplary compounds of the invention is prevented or retarded by the presence of an acid labile caging moiety, which, when cleaved, provides a compound that can be taken up by neoplastic tissue. An exemplary compound of the invention comprises a sugar moiety labeled with one or more radioisotopes of use in the diagnosis and/or treatment of disease. FIG. 2.

An exemplary compound of the invention has a structure according to Formula I:

A−(B)ₙ−C (I)

in which A is a sugar moiety functionalized with a radioisotope detectable by positron emission spectrometry. B is a linker, selected from a bond, a heteroatom or heteroatomic moiety, e.g., a hydrazone, oxime, glycosylamine, carbazone, and the like, substituted or unsubstituted alkyl, substituted or unsubstituted alkyl, e.g., benzyl, and a substituted or unsubstituted aryl or heteroaryl moiety. C is a caging moiety. Exemplary caging moieties are substituted or unsubstituted aryl and substituted or unsubstituted heteroaryl groups. The index n is 0 or 1, indicating the absence or presence of a linker, respectively, joining the sugar moiety and the caging moiety.

Exemplary sugar moieties include deoxy sugars in which one or more oxygen is replaced with one or more positron emitting atom. An exemplary sugar moiety is deoxyglucose, e.g., [¹⁸F]FDG. FIG. 4. An exemplary diagnostic agent of the invention takes advantage of the caging moiety to block the uptake of the radiotracer-labeled sugar moiety, e.g., [¹⁸F]FDG, by non-neoplastic tissue. In an exemplary embodiment, uptake of the radiotracer-labeled sugar moiety is prevented or retarded at neutral pH, but not at a pH more acidic than 7.4, e.g., less than pH 7, less than pH 6.5 or less than pH 6.0. FIG. 5.

Also provided are pharmaceutical formulations of the compounds of the invention in which the compound of the invention is dissolved in a pharmaceutically acceptable carrier. In various embodiments, the pharmaceutical formulation is a unit dosage formulation that includes a dosage of a compound of the invention appropriate for administration to a subject undergoing a diagnostic imaging procedure. In an exemplary embodiment, the unit dosage formulation is configured as a component of a kit. In an exemplary embodiment, a non-radioactive precursor of a compound of the invention is included in the kit. The kit optionally further includes one or more component of use to prepare a compound of the invention, formulate the compound of the invention and/or administer the compound of the invention to the subject undergoing a diagnostic imaging procedure.

In various embodiments, the invention provides a method of performing diagnostic imaging on a subject to whom the compound of the invention has been administered. The method includes, administering a diagnostically effective amount of a compound of the invention to the subject, and acquiring a diagnostic image of the subject, in which decay from the radioactive tracer of the invention is detected in at least one tissue of the subject. FIG. 6. In various embodiments, the compound of the invention provides access to a one step imaging modality for restaging patients with metastatic prostate cancer.

In an exemplary embodiment, the invention provides a data set acquired by performing an imaging method of the invention on a subject. The data set is generally an imaging data set. The data set can be stored on any form of readable physical media, can be uploaded to the internet, transmitted by telephony, etc.

In an exemplary embodiment, the invention provides one or more diagnostic compound for detecting and/or staging prostate cancer, a pharmaceutical formulation including such a compound, a method of using such a compound to detect and/or stage prostate cancer and an imaging data set acquired from a subject to who such a compound was administered.

### BRIEF DESCRIPTION OF THE FIGURES

FIG. 1 shows an array of tests used to diagnose and stage metastatic prostate cancer, and that no single imaging test suffices.

FIG. 2A and FIG. 2B show an exemplary acid labile FDG prodrug.

FIG. 3 shows solid tumors have acidic interstitial pH.

FIG. 4 shows exemplary FDGamines.

FIG. 5 shows FDGamines in vitro.

FIG. 6A and FIG. 6B show improved tumor to heart and tumor to brain ratio of FDGamine in comparison with FDG. FIG. 6A and FIG. 6B show PET imaging in PC3 xenografts demonstrating improved contrast to noise.

FIG. 7. FDGamine PET is of use to reduce the need for multiple imaging modalities.

FIG. 8. Commonly used acid-labile bonds.

FIG. 9. FDG delivered from cyclotron facility either mixed 1:1 with a 0.5 M solution of amine in water or DMSO OR FDG dried down at 110 degrees under vacuum/nitrogen stream and 0.5 M solution of amine added. Mixture incubated at 80 degrees for 30 minutes. Reaction purified on C18 HPLC, using 10 mM sodium phosphate pH 8.0 in running buffer to minimize decomposition. HPLC eluate diluted with water, loaded on C18 sep pak, washed with 10 mL water and sep pak eluted with 1 mL ethanol. Ethanol dried at RT under vacuum/nitrogen stream (about 30 min). Compound redissolved in saline for injection.

FIG. 10. HPLC degradation assay. Add 300 uL of HPLC purified compound to 2700 uL of 200 mM sodium phosphate pH 6.5 or 7.4, and incubate at 37 degrees. Analyze by analytical HPLC to separate prodrug from FDG.

FIG. 11. Exemplary amines of use in preparing the compounds of the invention and decomposition data for the compounds.

FIG. 12. PC3 cell uptake assay.

FIG. 13A and FIG. 13B. PET imaging studies: wild type mouse.

FIG. 14. PET dynamic vascular ROI analysis using an exemplary compound of the invention.

FIG. 15. PET dynamic vascular ROI analysis using an exemplary compound of the invention.

FIG. 16. PET region of interest analysis using an exemplary compound of the invention.

FIG. 17. PET region of interest analysis using an exemplary compound of the invention.

FIG. 18. PET region of interest analysis using an exemplary compound of the invention.

FIG. 19. Biodistribution (n=1) of an exemplary compound of the invention.

FIG. 20. Biodistribution (n=1) of an exemplary compound of the invention.

FIG. 21A. General structure of FDG-glycosylanilines of the invention.

FIG. 21B. Exemplary FDG-glycosylamines are synthesized in one step from ¹⁸F-FDG in indicated radiochemical yield.

FIG. 21C. The stability of exemplary FDG-glycosylamines were tested by incubation in phosphate buffer at the indicated pH, followed by radio-HPLC analysis.

FIG. 21D. Uptake of FDG-glycosylamines was assessed in a PC3 cell culture model by incubation in RPMI culture media at the indicated pH. Results are an average of 6 experiements±s.d.

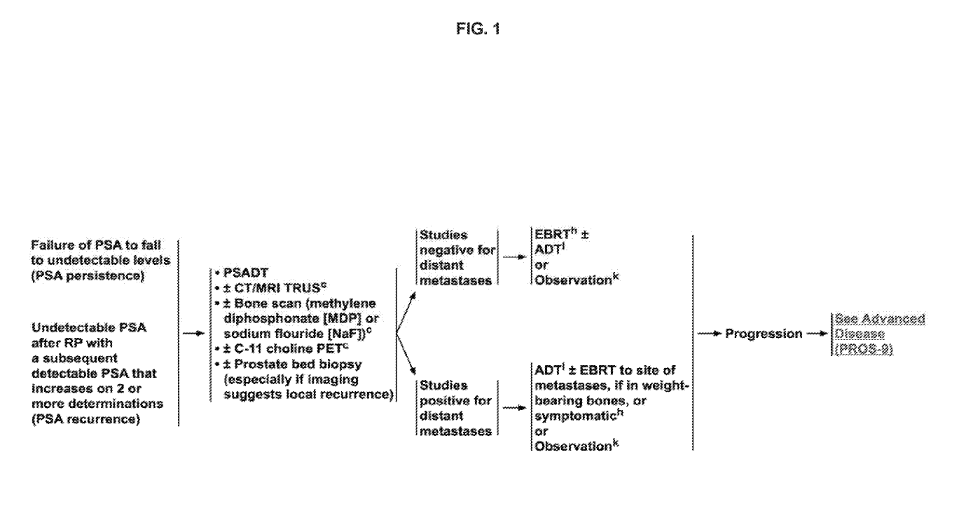

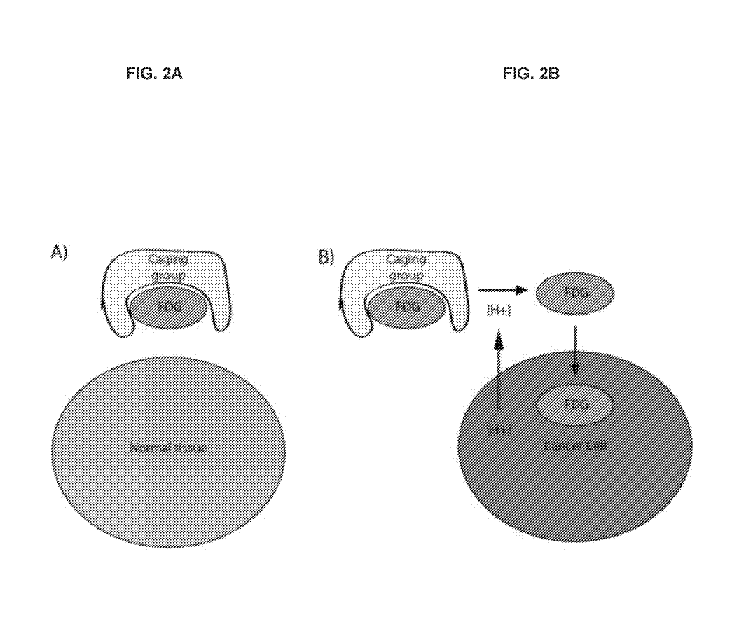

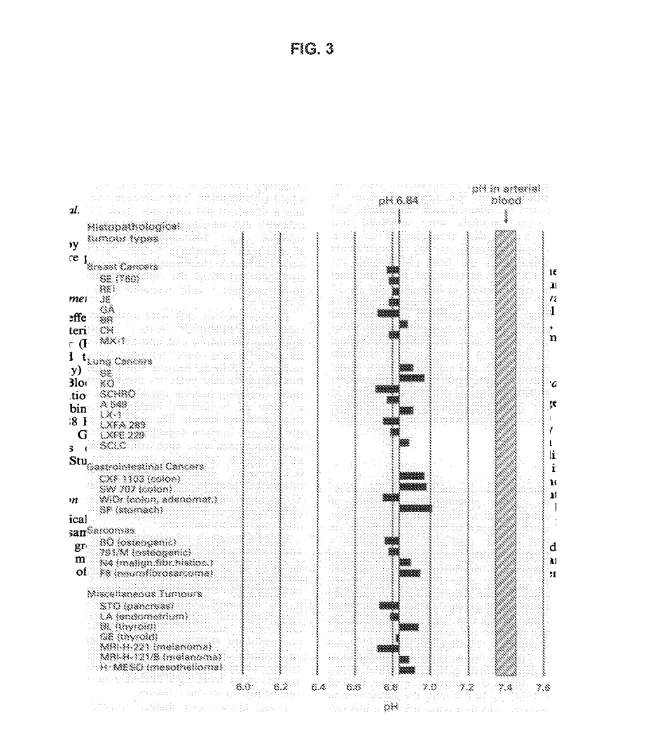

### DETAILED DESCRIPTION OF THE PREFERRED EMBODIMENTS

### Introduction

Solid tumors are hypoxic with altered metabolism, resulting in secretion of acids into the extracellular matrix and lower relative pH, a feature associated with local invasion and metastasis. Therapeutic and diagnostic agents responsive to this microenvironment may improve tumor-specific delivery. In various embodiments, the invention reflects a general strategy whereby caged small-molecule drugs or imaging agents liberate their parent compounds in regions of low interstitial pH. In this manuscript, we present a new acid-labile prodrug method based on the glycosylamine linkage, and its application to a class of positron emission tomography (PET) imaging tracers, termed [¹⁸F] fluorodeoxyglycosyl amines ([¹⁸F] FDG amines). [¹⁸F]FDG amines operate via a proposed two-step mechanism, in which an acid-labile precursor decomposes to form a common radiotracer, e.g., 2-deoxy-2-[18F]fluoro-D-glucose, which is subsequently accumulated by glycan, e.g., glucose, avid cells. The rate of decomposition of [18F]FDG amines is tunable in a systematic fashion, tracking the pKa of the parent amine. In vivo, a 4-phenylbenzylamine [18F]FDG amine congener showed greater relative accumulation in tumors over benign tissue, which could be attenuated upon tumor alkalinization using previously validated models, including sodium bicarbonate treatment, or overexpression of carbonic anhydrase. This new class of PET tracer represents a viable approach for imaging acidic interstitial pH with potential for clinical translation.

One in seven men will develop prostate cancer, and one in thirty five will die of the disease. In patients with progressive disease, there are a variety of treatment protocols and a long disease course, resulting in high imaging utilization.

The present invention relates to reaction based prodrugs of positron emission tomography (PET) probes that are sensitive to their environment and undergo cleaveage or another reaction in response to the environment. The reaction results in a change in the probe prodrug structure, and this change in structure is detectable by PET. The change in structure is indicative or confirmative of a property of the environment in which the probe prodrug of reacted prodrug is located. Environmental conditions that react with a prodrug of the invention include, without limitation, pH, hypoxia, or in general, the presence or absence of other analytes.

### II. Abbreviations

FDG is “fluorodeoxyglucose”.

### III Definitions

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

The compounds herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C=N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated.

The graphic representations of racemic, ambiscalemic and scalemic or enantiomerically pure compounds used herein are taken from Maehr, J. Chem. Ed., 62: 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but not implying any absolute stereochemistry; and wedge outlines and dotted or broken lines denote enantiomerically pure compounds of indeterminate absolute configuration.

The term “charged group” refers to a group that bears a negative charge or a positive charge. The negative charge or positive charge can have a charge number that is an integer selected from 1, 2, 3 or higher or that is a fractional number. Exemplary charged groups include for example —OPO₃²⁻, —P⁺Ph₃, —N⁺R′R″R″′, —S⁺R and —C(O)O⁻.

The compounds herein described may have one or more charged groups. For example, the compounds may be zwitterionic, but may be neutral overall. Other embodiments may have one or more charged groups, depending on the pH and other factors. In these embodiments, the compound may be associated with a suitable counter-ion. It is well known in the art how to prepare salts or exchange counter-ions. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Counter-ions may be changed, for example, by ion-exchange techniques such as ion-exchange chromatography. All zwitterions, salts and counter-ions are intended, unless the counter-ion or salt is specifically indicated. In certain embodiments, the salt or counter-ion may be pharmaceutically acceptable, for administration to a subject. Pharmaceutically acceptable salts are discussed later.

Alternatively, the compounds of the invention may form a compound including one or more charged groups following cleavage of the acid labile moiety.

The symbol [symbol] , whether utilized as a bond or displayed perpendicular to a bond indicates the point at which the displayed moiety is attached to the remainder of the molecule, solid support, etc.

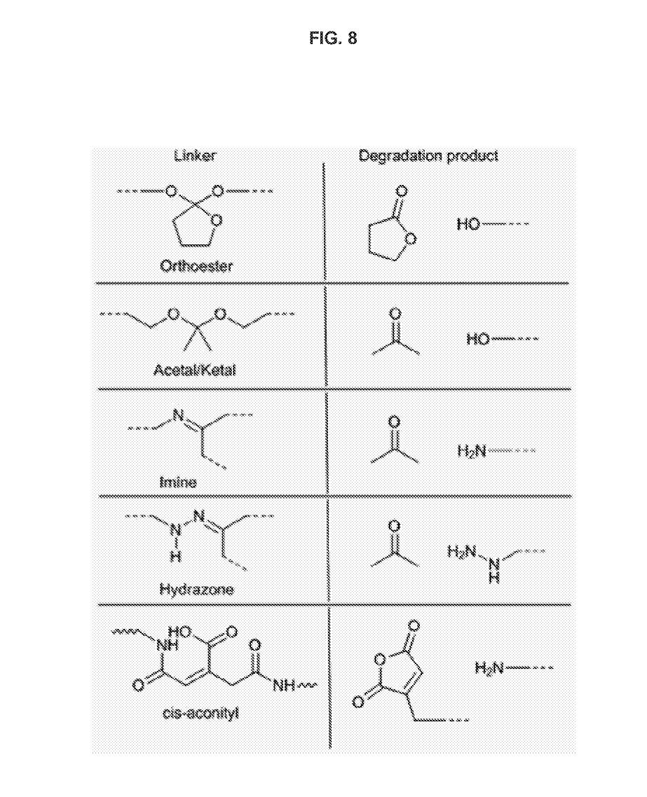

A “linker” as this term is used herein covalently joins the acid labile caging moiety to the caged radiotracer, or a species comprising radioisotope. Exemplary linkers include a bond (“zero-order”), substituted or unsubstituted alkyl, substituted or unsubstituted heteroalkyl, substituted or unsubstituted aryl, substituted or unsubstituted heteroaryl and substituted or unsubstituted heterocycloalkyl. Various exemplary linkers include C₁-C₁₀, e.g., C₁-C₆ substituted or unsubstituted alkyl or substituted or unsubstituted heteroalkyl moieties, and 5-7 member heterocycloalkyl residues, e.g., piperidine and piperazine. Exemplary linkers are set forth in FIG. 8.

When any variable occurs more than one time in any constituent or formula for a compound, its definition at each occurrence is independent of its definition at every other occurrence. Thus, for example, if a group is shown to be substituted with (X)ₙ, where n is 1, 2, 3, 4, or 5, then said group may optionally be substituted with up to five X groups and each occurrence is selected independently from the definition of X. Also, combinations of substituents and/or variables are permissible only if such combinations result in stable compounds.

As indicated above, various substituents of the various formulae are “substituted” or “may be substituted.” The term “substituted,” as used herein, means that any one or more hydrogens on the designated atom or group is replaced with a substituent, provided that the designated atom's normal valence is not exceeded, and that the substitution results in a stable compound. When a substituent is oxo (keto, i.e., =0), then 2 hydrogens on an atom are replaced. The present invention is intended to include all isotopes (including radioisotopes) of atoms occurring in the present compounds. When the compounds are substituted, they may be so substituted at one or more available positions, typically 1, 2, 3 or 4 positions, by one or more suitable groups such as those disclosed herein. Suitable groups that may be present on a “substituted” group include e.g., halogen; cyano; hydroxyl; nitro; azido; amino; alkanoyl (such as a C₁-C₆ alkanoyl group such as acyl or the like); carboxamido; alkyl groups (including cycloalkyl groups, having 1 to about 8 carbon atoms, for example 1, 2, 3, 4, 5, or 6 carbon atoms); alkenyl and alkynyl groups (including groups having one or more unsaturated linkages and from 2 to about 8, such as 2, 3, 4, 5 or 6, carbon atoms); alkoxy groups having one or more oxygen linkages and from 1 to about 8, for example 1, 2, 3, 4, 5 or 6 carbon atoms; aryloxy such as phenoxy; alkylthio groups including those having one or more thioether linkages and from 1 to about 8 carbon atoms, for example 1, 2, 3, 4, 5 or 6 carbon atoms; alkylsulfinyl groups including those having one or more sulfinyl linkages and from 1 to about 8 carbon atoms, such as 1, 2, 3, 4, 5, or 6 carbon atoms; alkylsulfonyl groups including those having one or more sulfonyl linkages and from 1 to about 8 carbon atoms, such as 1, 2, 3, 4, 5, or 6 carbon atoms; aminoalkyl groups including groups having one or more N atoms and from 1 to about 8, for example 1, 2, 3, 4, 5 or 6, carbon atoms; carbocyclic aryl having 4, 5, 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, (e.g. benzyl); arylalkoxy having 1 to 3 separate or fused rings and from 6 to about 18 ring carbon atoms (e.g. O-benzyl); or a saturated, unsaturated, or aromatic heterocyclic group having 1 to 3 separate or fused rings with 3 to about 8 members per ring and one or more N, O or S atoms, (e.g. coumarinyl, quinolinyl, isoquinolinyl, quinazolinyl, pyridyl, pyrazinyl, pyrimidyl, furanyl, pyrrolyl, thienyl, thiazolyl, triazinyl, oxazolyl, isoxazolyl, imidazolyl, indolyl, benzofuranyl, benzothiazolyl, tetrahydropyranyl, tetrahydropyranyl, piperidinyl, morpholinyl, piperazinyl, and pyrrolidinyl). Such heterocyclic groups may be further substituted, e.g. with hydroxy, alkyl, alkoxy, halogen and amino.

An “acid labile moiety”, as this term is used refers to a radical of a molecule of the invention that is removed from a compound of the invention at a pH below 7.4. When the acid labile group is present on the compound of the invention, its presence prevents or retards uptake of a compound containing such a moiety by proliferating cells.

Where substituent groups are specified by their conventional chemical formulae, written from left to right, the structures optionally also encompass the chemically identical substituents, which would result from writing the structure from right to left, e.g., —CH₂O— is intended to also optionally recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di-, tri- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to optionally include those derivatives of alkyl defined in more detail below, such as “heteroalkyl.” Alkyl groups that are limited to hydrocarbon groups are termed “homoalkyl”. Exemplary alkyl groups include the monounsaturated C₉₋₁₀, oleoyl chain or the diunsaturated C_{9-10, 12-13} linoeyl chain.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The terms “aryloxy” and “heteroaryloxy” are used in their conventional sense, and refer to those aryl or heteroaryl groups attached to the remainder of the molecule via an oxygen atom.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH=CH—O—CH₃, —Si(CH₃)₃, —CH₂—CH=N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Further exemplary cycloalkyl groups include steroids, e.g., cholesterol and its derivatives. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl substituent groups (or rings) that contain from one to four heteroatoms selected from N, O, S, Si and B, wherein the nitrogen and sulfur atoms are optionally oxidized, and the nitrogen atom(s) are optionally quaternized. An exemplary heteroaryl group is a six-membered azine, e.g., pyridinyl, diazinyl and triazinyl. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

As used herein, “heteroaryl” refers to a molecule having a reactive heteroaromatic core including one or more intra-annular nitrogen atoms. Exemplary heteroarenes are six-membered rings. This term encompasses diverse compounds, for example, substituted or unsubstituted pyridine, substituted or unsubstituted diazines (i.e., including two intra-annular nitrogen atoms), and substituted or unsubstituted triazines (i.e., including three intra-annular nitrogen atoms). Because heteroarene and heteroaryl describe, respectively, a parent molecule and a substituent formed from the parent molecule, these terms are not mutually exclusive terms and the structures of their reactive aromatic cores are co-extensive. Thus, the discussion regarding “heteroaryl” immediately above is germane to the definition of “heteroarene”.

For brevity, the term “aryl” when used in combination with other terms (e.g., aryloxy, arylthioxy, arylalkyl) includes aryl, heteroaryl and heteroarene rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl, “heteroaryl”, and “heteroarene”) are meant to optionally include both substituted and unsubstituted forms of the indicated species. Exemplary substituents for these species are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: H, substituted or unsubstituted aryl, substituted or unsubstituted heteroaryl, substituted or unsubstituted heterocycloalkyl, —OR′, ═O, ═NR′, ═N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R″′ and R′″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R″′ and R′″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like). These terms encompass groups considered exemplary “alkyl group substituents”, which are components of exemplary “substituted alkyl” and “substituted heteroalkyl” moieties.

Similar to the substituents described for the alkyl radical, substituents for the aryl heteroaryl and heteroarene groups are generically referred to as “aryl group substituents.” The substituents are selected from, for example: groups attached to the heteroaryl or heteroarene nucleus through carbon or a heteroatom (e.g., P, N, O, S, Si, or B) including, without limitation, substituted or unsubstituted alkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. Each of the above-named groups is attached to the heteroarene or heteroaryl nucleus directly or through a or a heteroatom (e.g., P, N, O, S, Si, or B); and where R′, R″, R″′ and R′″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″′ groups when more than one of these groups is present.

Two of the substituents on adjacent atoms of the aryl, heteroarene or heteroaryl ring may optionally be replaced with a substituent of the formula —T—C(O)—(CRR′)_{q}—U—, wherein T and U are independently —NR—, —O—, —CRR′- or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl, heteroarene or heteroaryl ring may optionally be replaced with a substituent of the formula —(CRR′)ₛ—X—(CR″R″′)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl. These terms encompass groups considered exemplary “aryl group substituents”, which are components of exemplary “substituted aryl” “substituted heteroarene” and “substituted heteroaryl” moieties.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non-aromatic rings. Examples of “fused ring systems” are naphthalenes, indoles, quinolines, chromenes and the like.

As used herein, the term “heteroatom” includes oxygen (O), nitrogen (N), sulfur (S) and silicon (Si) and boron (B).

The symbol “R” is a general abbreviation that represents a substituent group that is selected from H, substituted or unsubstituted alkyl, substituted or unsubstituted heteroalkyl, substituted or unsubstituted aryl, substituted or unsubstituted heteroaryl, and substituted or unsubstituted heterocycloalkyl groups.

In addition to a positron emitting atom, the compounds disclosed herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The phrase “pharmaceutically acceptable” is employed herein to refer to those compounds, materials, compositions, and/or dosage forms which are, within the scope of sound medical judgment, suitable for use in human beings and animals without excessive toxicity, irritation, allergic response, or other problem or complication, commensurate with a reasonable benefit/risk ratio.

The phrase “pharmaceutically acceptable carrier” as used herein means any pharmaceutically acceptable material, which may be liquid or solid. Exemplary carriers include vehicles, diluents, additives, liquid and solid fillers, excipients, solvents, solvent encapsulating materials. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention. “Compound or a pharmaceutically acceptable salt or solvate of a compound” intends the inclusive meaning of “or”, in that a material that is both a salt and a solvate is encompassed.

### Exemplary Embodiments

In various embodiments, the present invention provides compounds for detecting and/or quantifying cellular processes, diagnosing cancer and staging or restaging this disease using a PET detectable probe responsive to these cellular processes and tissue characteristics. Also provided are formulations containing these compounds, and methods of using the compounds in methods of diagnosis, staging and restaging of cancer.

In an exemplary embodiment, the positron emitting reaction-based probes of the invention include within their structure one or more moiety that is removed by an acidic environment or species. The present invention is further illustrated by reference to a representative class of probes that is activated (i.e., cleaved and entrapped within a cellular or tissue compartment) by cleaving the acid labile group, providing a uniform class of reaction-based probes.

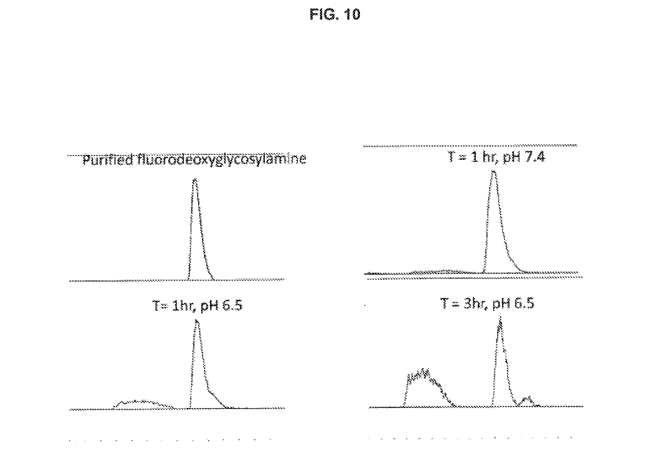

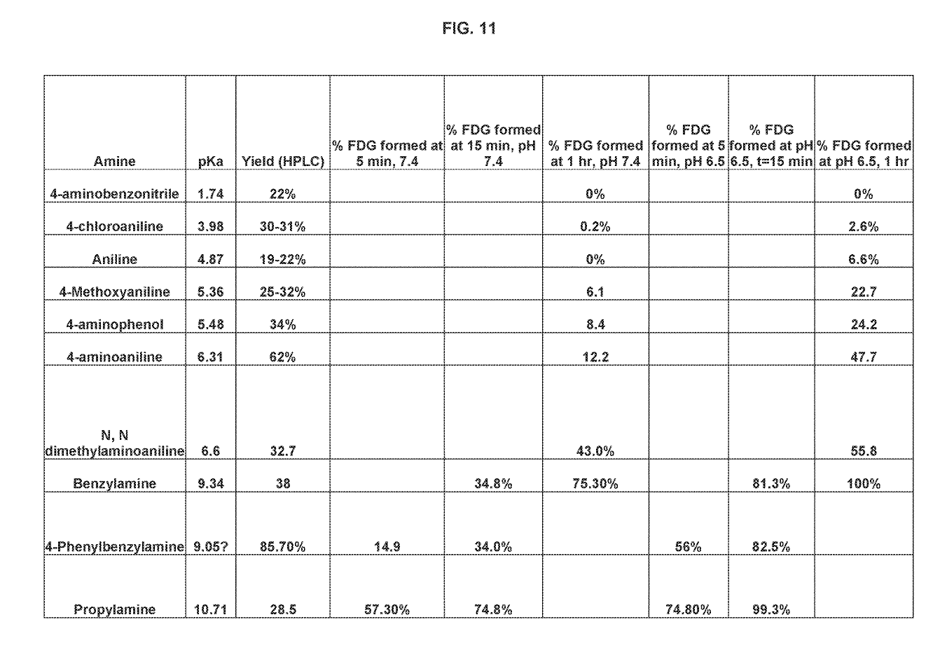

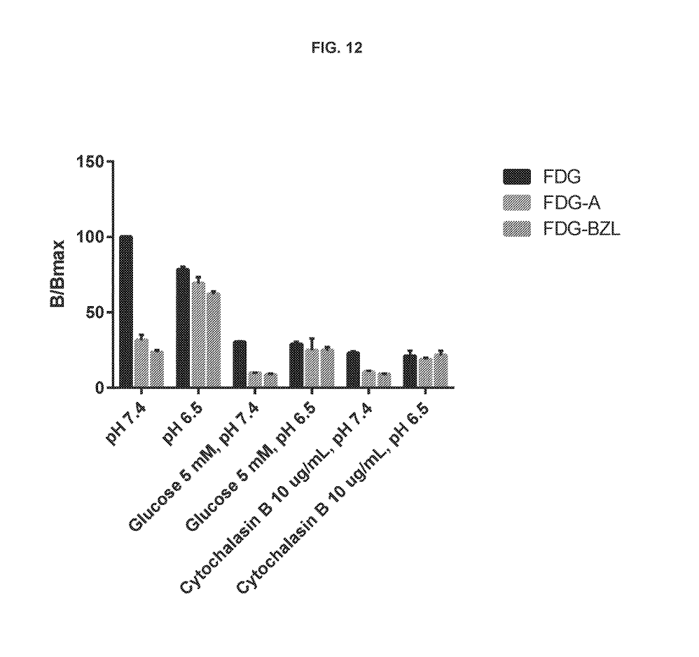

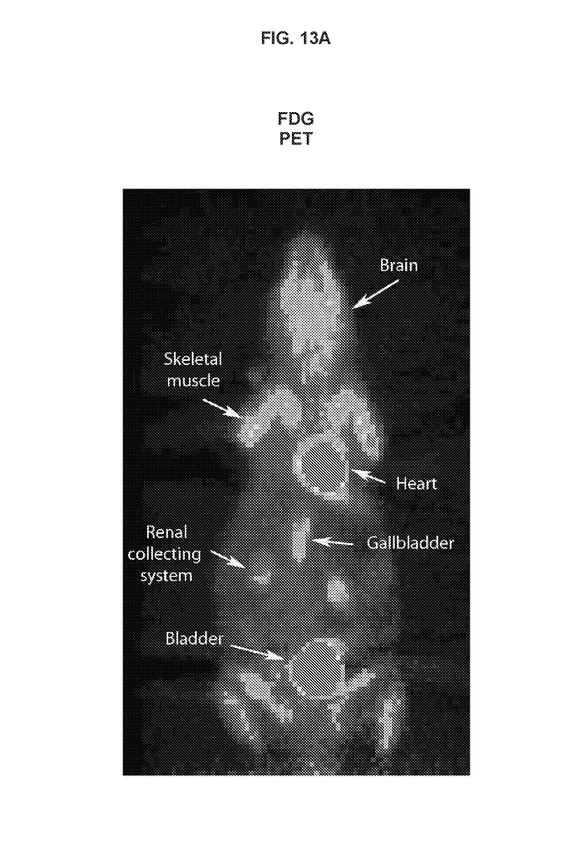

In an exemplary embodiment, the invention provides a reaction-based positron emission tomography probe for detection neoplastic tissue. The probe includes: (a) a positron emitting atom detectable by positron emission tomography. The positron emitting atom is covalently attached to; (b) a recognition moiety recognized and taken up by a proliferating cell or tissue, e.g., a sugar moiety. The recognition moiety is covalently attached to; (c) a caging moiety that is acid labile and, until it is cleaved from the recognition moiety, prevents or retards uptake of the recognition moiety by neoplastic cell or tissue. FIG. 10, FIG. 11. The caging moiety, in some embodiments, is attached to the recognition moiety by an acid labile linker. FIG. 8.

An exemplary recognition moiety is a mono-, or oligo-saccharide analogue (“sugar analogue”). In various embodiments, the carbon at the reducing terminus of the sugar analogue is derivatized with the caging moiety, though other positions on the sugar analogue can be labeled with the caging moiety, e.g., one or more of C-2, C3, C-4, C-5 and/or C-6. In various embodiments, one or more of these positions in addition to C-1 are functionalized with the caging moiety. In various embodiments, the hydroxyl at one or more of these positions is functionalized with the caging moiety.

Thus, in an exemplary embodiment, the invention provides a reaction-based positron emission tomography probe for detection of an acidic environment, such as that found in neoplastic tissue. The probe includes: (a) a positron emitting atom detectable by positron emission tomography. The positron emitting atom is covalently attached to; (b) a sugar analogue conjugated to an acid labile caging moiety that prevents or retards uptake of the probe by neoplastic tissue until the acid labile caging moiety is cleaved by contact with an acidic environment at which point the moiety on which the positron emitting moiety located is taken up by an adjacent metabolically active cell.

In an exemplary embodiment, the acid labile caging moiety is a substituted or unsubstituted aryl, e.g., substituted or unsubstituted aniline, substituted or unsubstituted heteroaryl, or substituted or unsubstututed alkyl, e.g., benzyl moiety.

In one embodiment, the radioactively labeled probe of the invention includes a sugar moiety labeled with a radioisotope. The probes are useful for detecting elevated cellular proliferation in vivo using positron emission tomography (PET). In an exemplary embodiment, the sugar is glucose or a deoxyglucose, e.g., 2-fluorodeoxyglucose.

In an exemplary embodiment, the labeled sugar analogues include a cleaveable acid labile moiety, comprising a caging moiety, cleaveable by acidic pH. The caging moiety masks an atom, e.g., a reducing hydroxyl of the sugar moiety.

In an exemplary embodiment, the caging moiety is a substituted or unsubstituted aryl or heteroaryl amine. Cleaveage of the compound of the invention provides an aniline and the positron emitting sugar analogue moiety.

An exemplary compound of the invention has a structure according to Formula I:

A—(B)ₙ—C (I)

in which A is a sugar moiety functionalized with a radioisotope detectable by positron emission spectrometry. B is a linker, selected from a bond, a heteroatom or heteroatomic moiety, e.g., a hydrazone, oxime, glycosylamines, carbozone, and the like, substituted or unsubstituted alkyl, and a substituted or unsubstituted heteroalkyl moiety. C is a caging moiety. Exemplary caging moieties include substituted or unsubstituted aryl, e.g., substituted or unsubstitued aniline, substituted or unsubstituted alkyl, e.g., benzyl, or substituted or unsubstituted heteroaryl groups. The index n is 0 or 1, indicating the absence or presence of a linker, respectively, joining the sugar moiety and the caging moiety.

In an exemplary embodiment, the linker, B, is selected from the moieties set forth in FIG. 8.

In an exemplary embodiment, the compound of the invention has the formula:

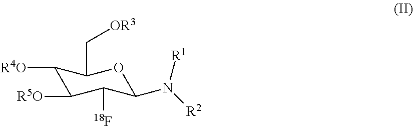

in which R¹ and R² are independently selected from H, substituted or unsubstituted aryl and substituted or unsubstituted heteroaryl, or substituted or unsubstituted alkyl, e.g., benzyl, with the proviso that at least one of these radicals is other than H, and at least one of these radicals is a caging moiety. R³-R⁵ are independently selected from H, substituted or unsubstituted alkyl, substituted or unsubstituted heteroalkyl, substituted or unsubstituted heterocycloalkyl, substituted or unsubstituted aryl and substituted or unsubstituted heteroaryl.

In an exemplary embodiment, at least one of R¹ and R² is a caging moiety having the formula:

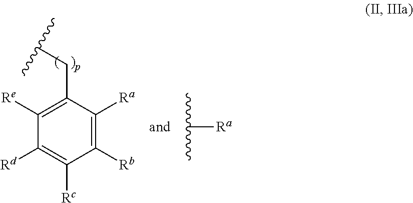

in which R^{a}-R^{e} are members independently selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁷R⁸, —NR⁷R⁸, —OR′, —S(O)₂R⁷, —C(O)R⁷, —COOR⁷, —CONR⁷R⁸, —S(O)₂OR⁷, —OC(O)R⁷, —C(O)NR⁷R⁸, —NR⁷C(O)R⁸, —NR⁷SO₂R⁸ and —NO₂, wherein two or more of R^{a}, R^{b}, R^{c}, R^{d} and R^{e}, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl. The index p is an integer selected from 0, 1, 2, 3, 4, 5, 6, or higher.

The symbols R⁷ and R⁸ represent members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁷ and R⁸, together with the atoms to which they are bonded, are optionally joined to form a 5- to 7-membered ring which is a member selected from substituted or unsubstituted cycloalkyl, substituted or unsubstituted heterocycloalkyl, substituted or unsubstituted aryl and substituted or unsubstituted heteroaryl.

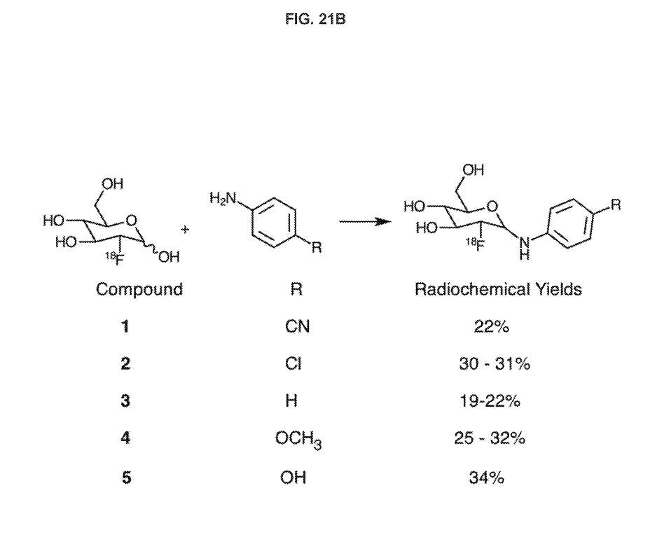

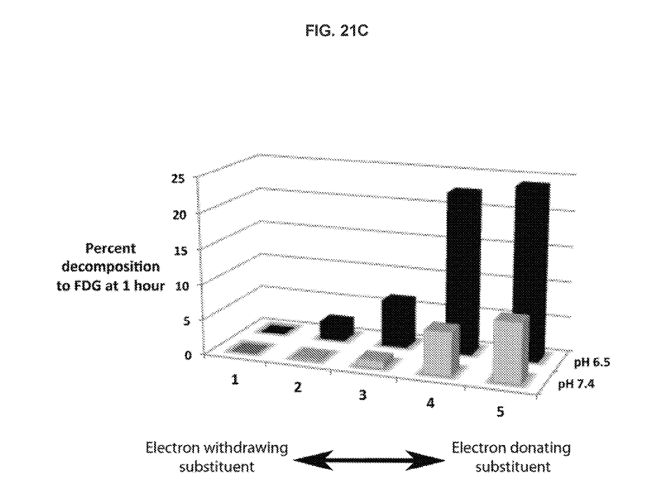

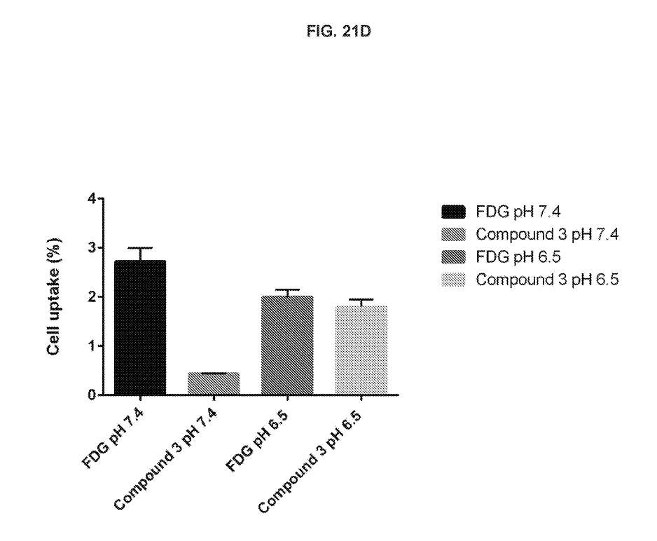

In an exemplary embodiment, the caging moiety is selected from the amines set forth in FIG. 11 and FIG. 21b. As one of skill in the art will appreciate, placing the caging moiety involves loss of a hydrogen from the amines listed in FIG. 11. FIG. 21b shows exemplary reaction schemes for making the compounds of the invention.

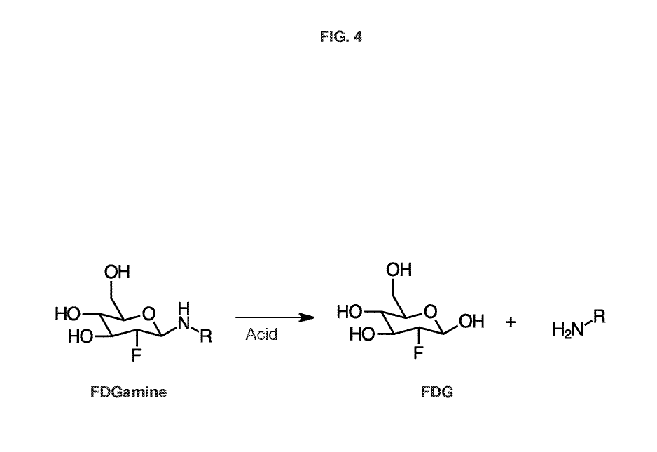

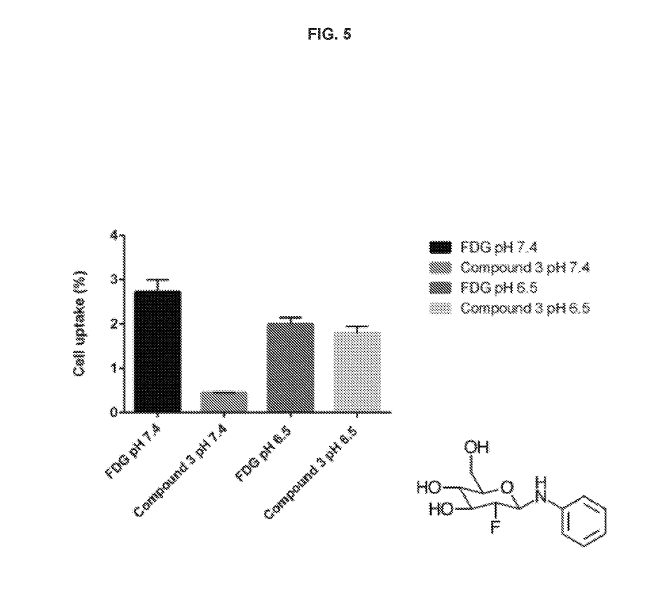

In one embodiment, the sugar analogs are deoxyglucose analogues bearing an amine on the carbon at position 1 of the sugar moiety (FIG. 4 and FIG. 5). In various embodiments, the amine is aniline or a substituted aniline.

In one embodiment, the structural components of these novel sugar analogues, include the sugar scaffold and a linker. In one variation, the structural components of the compounds of the invention include a sugar, an acid labile linker and a linker and a radioactive isotope such as ¹⁸F-fluorine or ¹¹C-carbon covalently bound to the sugar.

In one embodiment, the caged probe species of the present invention is cell impermeant, and becomes cell permeant upon cleaveage of the acid labile caging moiety.

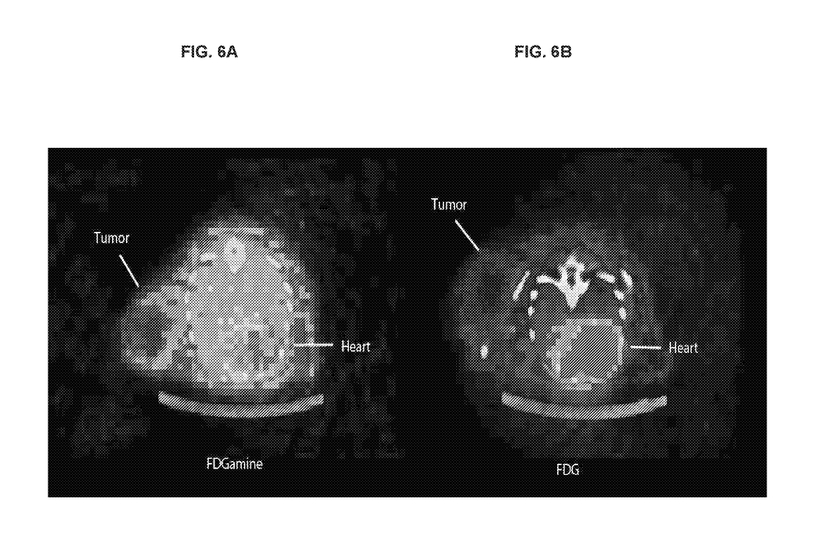

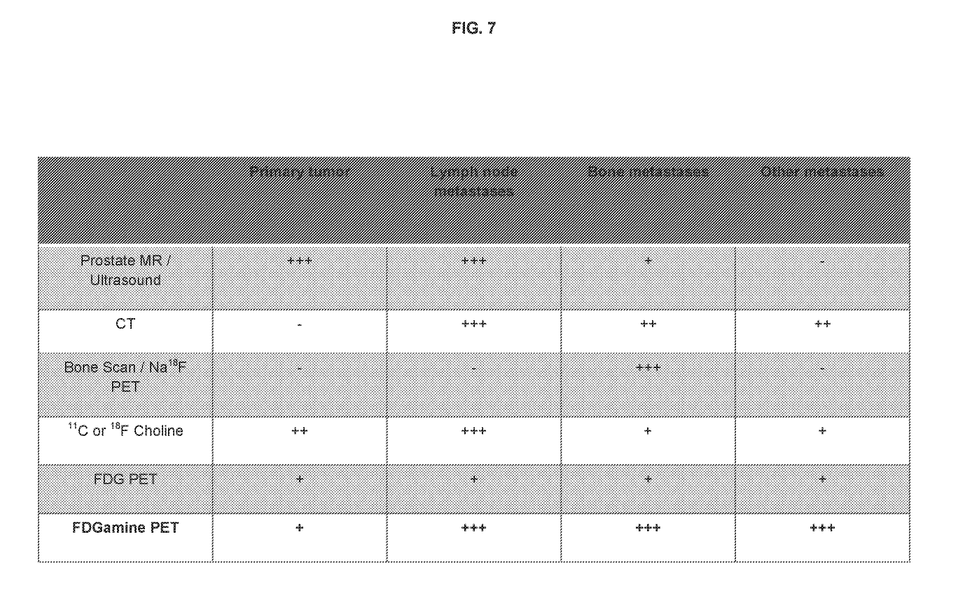

In various embodiments, the invention provides probes that become entrapped within a cellular or tissue compartment upon their conversion to an uncaged species. An exemplary probe of the invention is uncaged by acid and subsequently intracellularly entrapped. The entrapped probe can be utilized to observe and stage the neoplastic tissue in the region of interest by using standard positron emission tomography (PET) techniques. See, e.g., FIG. 6.

In various embodiments, the recognition moiety and caging moiety are attached by a linker. Resentative linkers include, for example, substituted or unsubstituted alkyl groups, substituted heteroalkyl groups, conjugated unsaturated systems, aryl groups, heteroaryl groups, dendrimers, polyethers, polyamides, polyimines, biopolymers and linkers that are a combination of more than one of these groups. Other useful linkers will be apparent to those of skill in the art. The linker is generally attached to a component of the compound of the invention (or its positron emitting analogue) through a linking group formed through reaction between a reactive group on the cellular recognition moiety precursor and a complementary reactive group on a linker arm precursor. The linker is attached to a carrier species through a similar reactive group.

Exemplary linkers include a bond (“zero-order”), substituted or unsubstituted alkyl, substituted or unsubstituted heteroalkyl, substituted or unsubstituted aryl, substituted or unsubstituted heteroaryl and substituted or unsubstituted heterocycloalkyl. Various exemplary linkers include C₁-C₁₀, preferably, C₁-C₆ substituted or unsubstituted alkyl or substituted or unsubstituted heteroalkyl moieties, and 5-7 member heterocycloalkyl residues, e.g., piperidine and piperazine.

In an exemplary embodiment, the caging moiety is cleaved from the rest of the molecule (i.e., the sugar moiety or the linker/sugar moiety cassette) under acidic conditions. In an exemplary embodiment, the the caging moiety is cleaved from the rest of the molecule at a pH of about 6.5. In various embodiments, the structure of the molecule is selected such that the more of the caging moiety is cleaved from the rest of the molecule at a pH of about 6.5 than is cleaved at a pH of about 7.4 within the same unit of time.

Moreover, the compounds of the invention can be connected to a carrier species, e.g., biomolecule, by a linker of substantially any length (zero-order or higher) and chemical composition.

Other embodiments provide kits comprising a compound according to the invention. In certain embodiments, the kit provides packaged pharmaceutical compositions comprising a pharmaceutically acceptable carrier and a compound of the invention. In certain embodiments the packaged pharmaceutical composition will comprise the reaction precursors necessary to generate the compound of the invention upon combination with a radiolabeled precursor. Other packaged pharmaceutical compositions provided by the present invention further comprise indicia comprising at least one of: instructions for preparing compounds according to the invention from supplied precursors, instructions for using the composition to image neoplastic cells or tissues, or instructions for using the composition to image prostate or other cancers.

In certain embodiments, a kit according to the invention contains from about 1 to about 30 mCi of the radionuclide-labeled imaging agent described above, in combination with a pharmaceutically acceptable carrier. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like. The kit may provide a compound of the invention in solution or in lyophilized form, and these components of the kit of the invention may optionally contain stabilizers such as NaCl, silicate, phosphate buffers, ascorbic acid, gentisic acid, and the like. Additional stabilization of kit components may be provided in this embodiment, for example, by providing the reducing agent in an oxidation-resistant form. Determination and optimization of such stabilizers and stabilization methods are well within the level of skill in the art.

In various embodiments, the invention provides a pharmaceutical formulation comprising one or more compound of the invention in combination with a pharmaceutically acceptable carrier.

### Synthesis

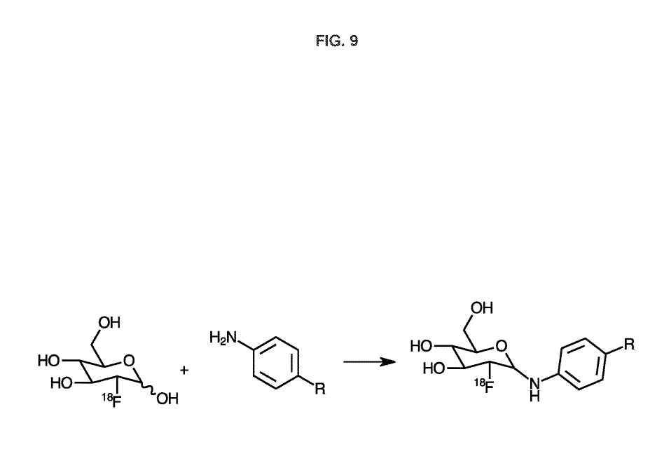

The compounds of the invention are synthesized by an appropriate combination of generally well-known synthetic methods. Techniques useful in synthesizing the compounds of the invention are both readily apparent and accessible to those of skill in the relevant art. The discussion below is offered to illustrate certain of the diverse methods available for use in assembling the compounds of the invention, it is not intended to limit the scope of reactions or reaction sequences that are useful in preparing the compounds of the present invention. FIG. 9.

The compounds of the invention can be prepared as a single stereoisomer or as a mixture of stereoisomers. In a preferred embodiment, the compounds are prepared as substantially a single isomer. Isomerically pure compounds are prepared by using synthetic intermediates that are isomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose an appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

### Methods

The present invention also provides methods of using the compounds described herein to detect neoplastic tissue in a subject or sample. The methods are illustrated by the use of the compound of the invention to detect prostate cancer. Those of skill in the art will appreciate that this focus is for clarity of illustration and does not limit the scope of the methods in which the compounds of the invention find use.

In an exemplary embodiment, the probe of the invention is utilized in PET imaging of a subject to whom is administered a diagnostically effective amount of the probe.

As described above, the radiolabeled probes are useful for imaging cellular proliferation in a subject. When administered to a subject, the radiolabeled probes can be administered as a component of a pharmaceutical formulation including a pharmaceutically acceptable carrier or vehicle. The present compositions, which comprise a radiolabeled probe, can be administered by any convenient route, for example, by infusion, bolus injection, or by absorption through epithelial or mucocutaneous linings and can be administered together with another biologically active agent. Administration can be systemic or local. Methods of administration include, but are not limited to, intradermal, intramuscular, intraperitoneal, intravenous, subcutaneous, intranasal, sublingual, epidural, intracerebral, intravaginal, transdermal, rectal, or topical.

The radiolabeled probes may be administered locally. This can be achieved, for example, and not by way of limitation, by local infusion during surgery, by injection, by means of a catheter, by means of a suppository or enema, or by means of an implant, with said implant being of a porous, non-porous, or gelatinous material, including membranes, such as sialastic membranes or fibers. In addition, the radiolabeled probes can be delivered in a controlled-release system or sustained-release system. The controlled-release system or sustained-release system can be placed in proximity to a target of the radiolabeled probes, e.g., the spinal column, brain, heart, kidney or gastrointestinal tract, thus requiring only a fraction of the systemic dose.

The compositions can optionally comprise a suitable amount of a physiologically acceptable excipient so as to provide the form for proper administration to the subject. Such physiologically acceptable excipients can be liquids, such as water for injection, bactereostatic water for injection, or sterile water for injection. The physiologically acceptable excipients are sterile when administered to a subject. Water is a particularly useful excipient when the radiolabeled triazole compound is administered intravenously. Saline solutions can also be employed as liquid excipients, particularly for injectable solutions. The pharmaceutical excipients can be saline, gum acacia, starch, glucose, lactose, glycerol, ethanol and the like.

Moreover, the radiolabeled probes can be formulated for intravenous administration. Typically, compositions for intravenous administration comprise sterile isotonic aqueous buffer. Where the radiolabeled probes are administered by injection, an ampule of sterile water for injection or saline can be provided so that the ingredients can be mixed prior to administration. Where the radiolabeled probes are to be administered by infusion, they can be dispensed, for example, with an infusion bottle containing sterile pharmaceutical grade water or saline.

The amount of the radiolabeled probe that is effective as an imaging agent to detect cellular proliferation in a subject can be determined using standard clinical and nuclear medicine techniques. In addition, in vitro or in vivo testing can optionally be employed to help identify optimal dosage ranges. The precise dose to be employed will also depend on the route of administration, the identity of the subject and the identity of the particular radionuclide being detected and should be decided according to the judgment of the practitioner and each subject's circumstances in view of published clinical studies.

The probe may be administered in a unit dosage form, such that a unit dose of the imaging agent is a non-toxic amount of the probe (e.g., the nucleoside analogue) capable of localizing in proliferating cells and being detected in vivo. The specific activities of the probe prepared as described herein below will generally range from about 1.5 to about 2.0 curies/micromole (Ci/μmol). In an exemplary embodiment, the positron emitting atom is ¹⁸F. Given that ¹⁸F has a half life of about 110 minutes, a unit dose of may be in the range from about 100 to about 200 microcuries (μCi) of the ¹⁸F labeled probe, for example. (For comparison with new standards using Becquerel units (Bq), 37 Bq=1 nanocurie, thus there are 37 KBq to 1 microcurie.)

The present invention also provides a method for in vivo diagnostic imaging of cellular proliferation which includes administering to a subject in need thereof a probe of the invention radiolabeled. The probe may be administered in accordance with procedures known in the art. For example, about 100 to about 200 μCi of radiolabeled material in physiological saline solution or equivalent vehicle along with any necessary adjuvant and other pharmaceutically acceptable carriers is administered intravenously to a subject prior to imaging or probe studies.

Detecting the positron emitting probe in vivo localized in proliferating cells is carried out by standard procedures. Data collection following administration may involve dynamic or static techniques with a variety of imaging devices, including PET cameras, gamma or SPECT (single photon emission computed tomography) cameras with either high energy collimators or coincidence detection capabilities, and probe devices designed to measure radioactive counts over specific regions of interest.

In an exemplary embodiment, the present invention provides methods of using the compounds described herein to detect an analyte in a sample, e.g., a neoplastic tissue or cell. Alternatively, the present compounds are also used to detect or monitor distribution of neoplastic cells.

In various embodiments, there is provided a method for determining the presence or absence of an acidic environment (e.g., due to the presence of neoplastic tissue) in a sample. The method includes: a) contacting the sample with a probe compound having a structure according to Formula I; b) incubating the labeled sample for a sufficient amount of time to allow interaction between the probe and the acidic environment to produce an uncaged product; c) observing the presence or absence of positron emission from the sample, whereby the presence or absence of the probe in the sample is determined.

In other embodiments, the compounds according to Formula I are utilized to stain a sample to give a detectable positron emission response under desired conditions by first preparing a probe solution comprising a probe compound described above, at a concentration sufficient to yield a detectable positron emission response under the desired conditions. Specifically the methods for staining a sample include: a) contacting the sample with a probe having a structure according to Formula I; b) incubating the stained sample for a sufficient amount of time to allow reaction between the probe and an acidic milieu, producing an entrapped probe; and c) detecting positron emission in the sample.

For example, the entrapped probe derived from a species of the invention is used to monitor specific components of the sample with respect to their spatial and temporal distribution in the sample. Alternatively, the entrapped probe preferentially binds to a specific analyte in a sample, enabling the researcher to determine the presence or quantity of that specific analyte. In another embodiment, the compound of the invention is used to analyze the sample for the presence of a mechanism that acts upon the entrapped probe, e.g., oxidation or reduction. The desired analysis to be performed determines the composition of the probe solution (formulation) and chemical nature of the probe itself.

In one embodiment, the caged probe species of the present invention is substantially cell impermeant, and can be introduced into the sample cell or cells by microinjection of a solution of the probe, scrape loading techniques (short mechanical disruption of the plasma membrane where the plasma membrane is peeled away from the cytoplasm, the probe is perfused through the sample and the plasma membrane reassembled), or patch clamp methods (where an opening is maintained in the plasma membrane for long periods) can be used. Any other treatment that will permeabilize the plasma membrane, such as electroporation, shock treatments or high extracellular ATP can be used to accelerate introduction of the probe into the cellular cytoplasm. Microinjection of a probe solution is of particular use when analysis of a single cell is desired, within a colony of other sample cells.

Other embodiments of the inventions include methods of imaging one or more cells, organs or tissues comprising exposing cells to or administering to a subject an effective amount of a compound of the invention with an isotopic label suitable for imaging. In some embodiments, the one or more organs or tissues include prostate tissue, kidney tissue, brain tissue, vascular tissue or tumor tissue.

In another embodiment, the imaging method is suitable for imaging studies of chemotherapeutic agents, for example, by studying competitive binding of non-radiolabeled inhibitors. In still another embodiment, the imaging method is suitable for imaging of cancer, tumor or neoplasm. In a further embodiment, the cancer is selected from eye or ocular cancer, rectal cancer, colon cancer, cervical cancer, prostate cancer, breast cancer and bladder cancer, oral cancer, benign and malignant tumors, stomach cancer, liver cancer, pancreatic cancer, lung cancer, corpus uteri, ovary cancer, prostate cancer, testicular cancer, renal cancer, brain cancer (e.g., gliomas), throat cancer, skin melanoma, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, neurofibromatosis, tuberous sclerosis, hemangiomas, and lymphangiogenesis. Methods of the present invention can be used to image nearly all solid tumors including lung, renal cell, glioblastoma, pancreas, bladder, sarcoma, melanoma, breast, colon, germ cell, pheochromocytoma, esophageal and stomach. Also, certain benign lesions and tissues including endometrium, schwannoma and Barrett's esophagus can be imaged according to the present invention.

The methods of imaging angiogenesis provided by the present invention are suitable for use in imaging a variety of diseases and disorders in which angiogenesis takes place. Illustrative, non-limiting, examples include tumors, collagen vascular disease, cancer, stroke, vascular malformations, retinopathy. Methods of imaging angiogenesis provided by the present invention are also suitable for use in diagnosis and observation of normal tissue development.

In certain embodiments, the radiolabeled compound is detected by positron emission tomography (PET) or single photon emission computed tomography (SPECT).

In one embodiment, the invention provides a method wherein the subject is a human, rat, mouse, cat, dog, horse, sheep, cow, monkey, avian, or amphibian. In another embodiment, the cell is in vivo or in vitro. Typical subjects to which compounds of the invention may be administered will be mammals, particularly primates, especially humans. For veterinary applications, a wide variety of subjects will be suitable, e. g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications.

In certain methods of the invention the compounds of the invention are excreted from tissues of the body quickly to prevent prolonged exposure to the radiation of the radiolabeled compound administered to the patient. Typically compounds of the invention are eliminated from the body in less than about 24 hours. More typically, compounds of the invention are eliminated from the body in less than about 16 hours, 12 hours, 8 hours, 6 hours, 4 hours, 2 hours, 90 minutes, or 60 minutes. Exemplary compounds are eliminated in between about 60 minutes and about 120 minutes.

In certain embodiments, the positron emitting moiety (e.g., sugar analogue) of the invention are stable in vivo such that substantially all, e.g., more than about 50%, 60%, 70%, 80%, or 90% of the injected compound is not metabolized by the body prior to excretion. Typical subjects to which compounds of the invention may be administered will be mammals, particularly primates, especially humans.

In an exemplary embodiment, the invention provides an imaging method for single step restaging of cancer, e.g., prostate cancer, e.g., metastatic prostate cancer.

Other embodiments of the invention provide methods of treating tumors comprising administering to a subject a therapeutically effective amount of a compound according to the present invention comprising a therapeutically effective radioisotope. In certain embodiments, the tumor cells are prostate tumor cells or metastasized prostate tumor cells.

In various embodiments, the invention provides a method of using PET to study tumor associated inflammation.

In various embodiments, the invention provides a single step restaging of metastatic prostate cancer.

For veterinary applications, a wide variety of subjects will be suitable, e.g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications.

Imaging agents of the invention may be used in accordance with the methods of the invention by one of skill in the art. Images can be generated by virtue of differences in the spatial distribution of the imaging agents which accumulate at a site. The spatial distribution may be measured using any means suitable for the particular label, for example, a gamma camera, a PET apparatus, a SPECT apparatus, and the like. The extent of accumulation of the imaging agent may be quantified using known methods for quantifying radioactive emissions. A particularly useful imaging approach employs more than one imaging agent to perform simultaneous studies.

In general, a detectably effective amount of the imaging agent of the invention is administered to a subject. In accordance with the invention, “a detectably effective amount” or “diagnostically effective amount” of the imaging agent of the invention is defined as an amount sufficient to yield an acceptable image using equipment which is available for clinical use. A detectably effective amount of the imaging agent of the invention may be administered in more than one injection. The detectably effective amount of the imaging agent of the invention can vary according to factors such as the degree of susceptibility of the individual, the age, sex, and weight of the individual, idiosyncratic responses of the individual, and the dosimetry. Detectably effective amounts of the imaging agent of the invention can also vary according to instrument and film-related factors. Optimization of such factors is well within the level of skill in the art. The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

### Sample Preparation

The compounds of the invention are also of use in in vitro assays of samples of interest. The end user will determine the choice of the sample and the way in which the sample is prepared. The sample includes, without limitation, any biological derived material or aqueous solution that is thought to contain a target analyte, peroxide or an enzymatic system that produces peroxide. The samples may also include a reactive oxygen species, e.g., peroxide, or a molecule or system, e.g., an enzymatic system that produces peroxide. Furthermore, the sample can include a buffer solution that contains a peroxidase, peroxide and probe compounds of the present invention to determine the ability of the sample to oxidize the compound of the invention.

The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells and eukaryotic cells that include primary cultures and immortalized cell lines. Eukaryotic cells include without limitation ovary cells, epithelial cells, circulating immune cells, (3 cells, hepatocytes, and neurons.

Various buffers may be used. By way of illustration, these buffers include PBS, Tris, MOPS, HEPES, phosphate, etc. The pH will vary depending upon the particular monooxygenase being assayed, generally being in the range of about 7.0-7.5, where the pH is selected to provide for at least about maximum enzyme activity. The concentration of buffer will be sufficient to prevent a significant change in pH during the course of the reaction, generally being in the range of about 0.1 to 100 mM, more usually 0.5 to 50 mM.

The reaction time will usually be at least about 5 min, more usually at least about 30 min and preferably not more than about 120 min, depending upon the temperature, concentrations of enzyme and substrate, etc. By using a specific time period for the reaction or measuring the positron emission at 2 different times, the rate of reaction can be determined for comparison with other determinations. The temperature will generally be in the range of about 20 to 50° C., more usually in the range of about 25 to 40° C.

In certain instances, it may be advantageous to add a small amount of a non-ionic detergent to the sample. Generally the detergent will be present in from about 0.01 to 0.1 vol. %. Illustrative non-ionic detergents include the polyoxyalkylene diols, e.g. Pluronics, Tweens, Triton X-100, etc.

After sufficient time for a detectable amount of product to form, the reaction is optionally quenched. Various quenching agents may be used, both physical and chemical. Conveniently, a small amount of a water-soluble inhibitor may be added, such as acetonitrile, DMSO, SDS, methanol, DMF, etc. The amount of inhibitor will vary with the nature of the inhibitor and may be determined empirically.

The present invention may be better understood by reference to the following non-limiting Examples, which are provided as exemplary of the invention. The following examples are presented in order to more fully illustrate preferred embodiments of the invention, but should in no way be construed as limiting the broad scope of the invention.

While this invention has been disclosed with reference to specific embodiments, it is apparent that other embodiments and variations of this invention may be devised by others skilled in the art without departing from the true spirit and scope of the invention.

All patents, patent applications, and other publications cited in this application are incorporated by reference in the entirety.

### EXAMPLES

### Example 1

An example of synthesis of aniline-FDG amine is provided, FIG. 9, R=H. ¹⁸F-Fluorodeoxyglucose provided by standard radiosynthetic technique (typically 50-500 μL, 5-100 mCi) was dried azeotropically using acetonitrile at 110° C. under vacuum and nitrogen stream. To this was added 250 μL of a solution containing 0.5M Aniline-HCl, 0.5M sodium acetate. The reaction was incubated at 80° C. for 30 minutes. The reaction was then purified using semipreperative high performance liquid chromatography (HPLC) to separate from unreacted starting materials. The purified probe was then reformulated into normal saline for injection. The probe was characterized by co-injection on analytical HPLC with the corresponding structurally characterized, non-radiolabeled standard compound.

### Example 2

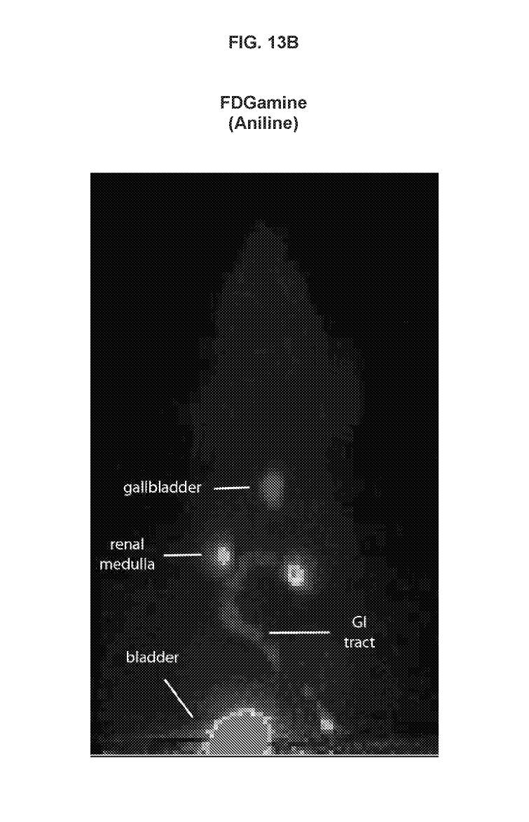

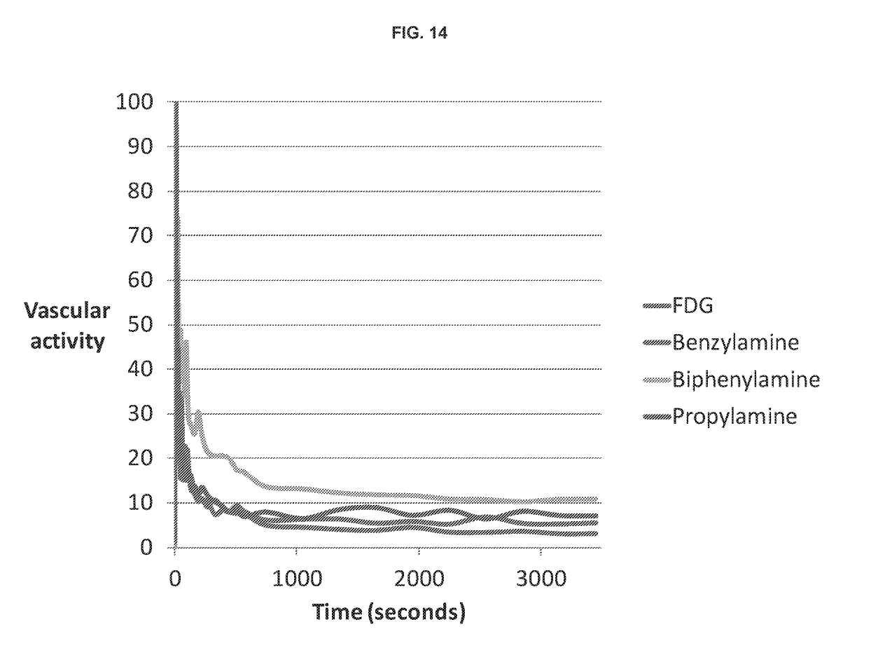

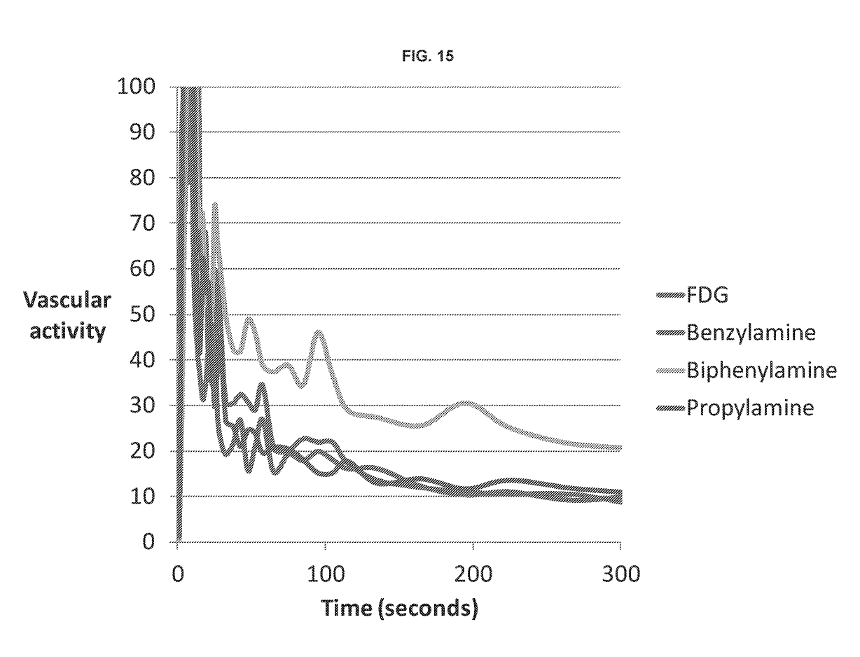

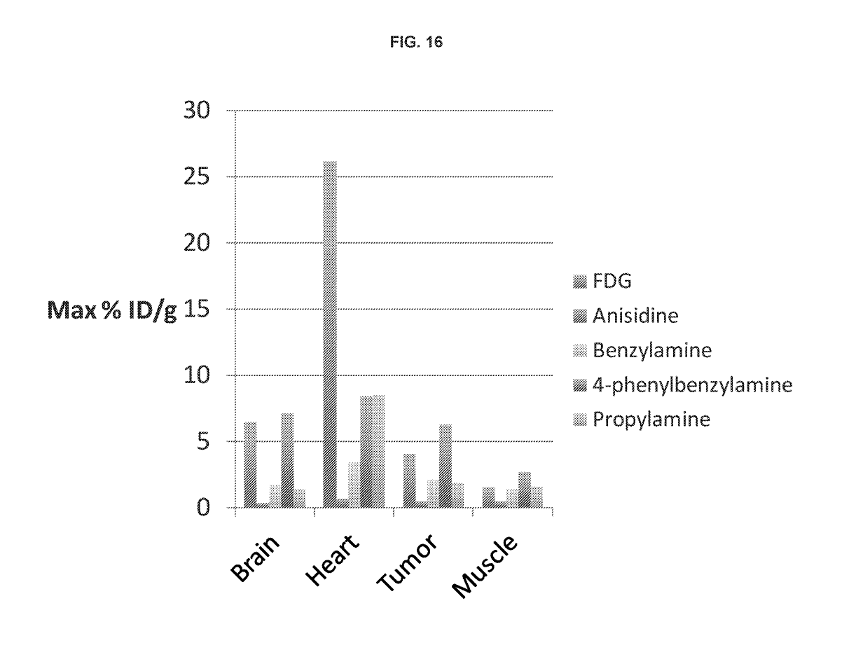

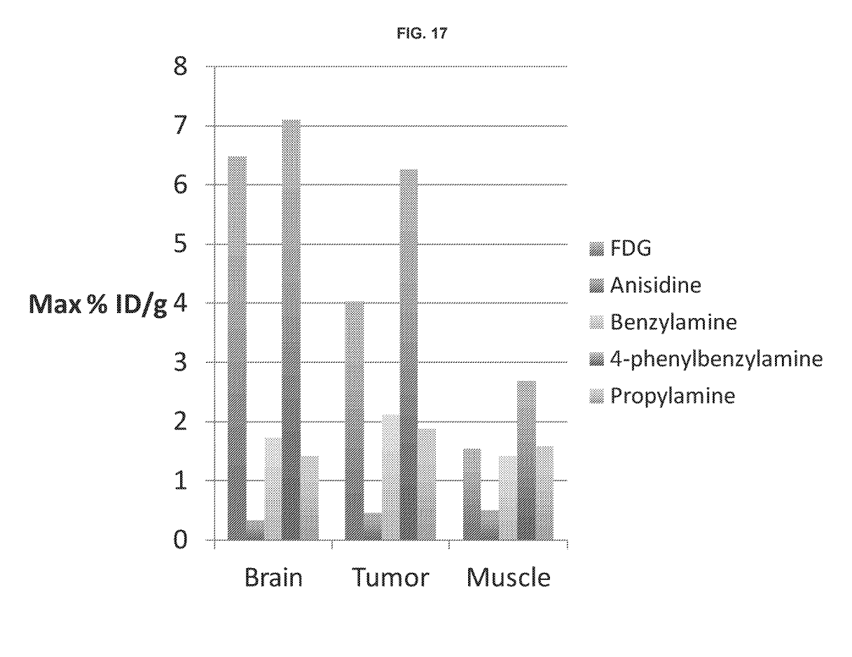

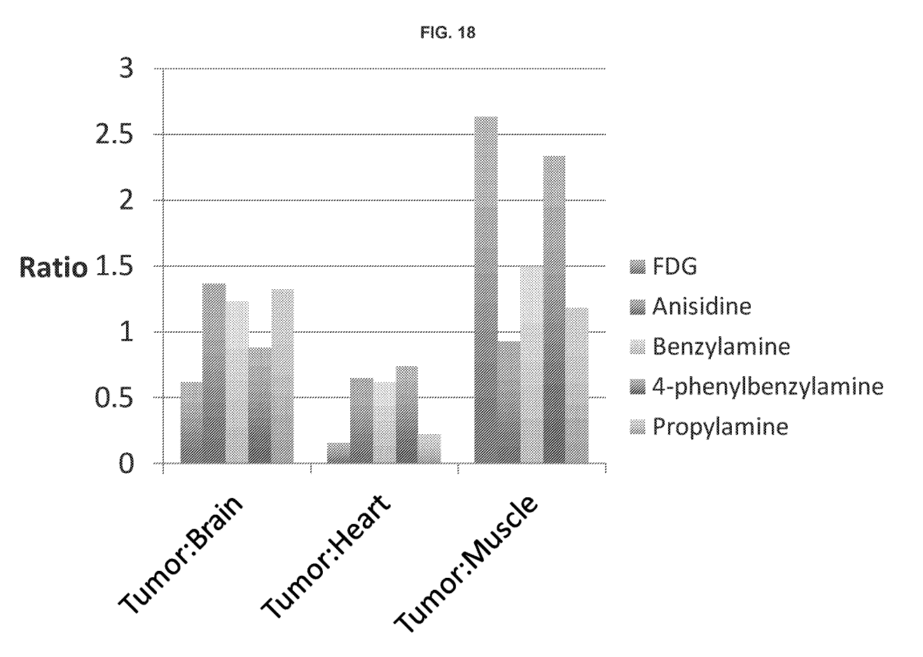

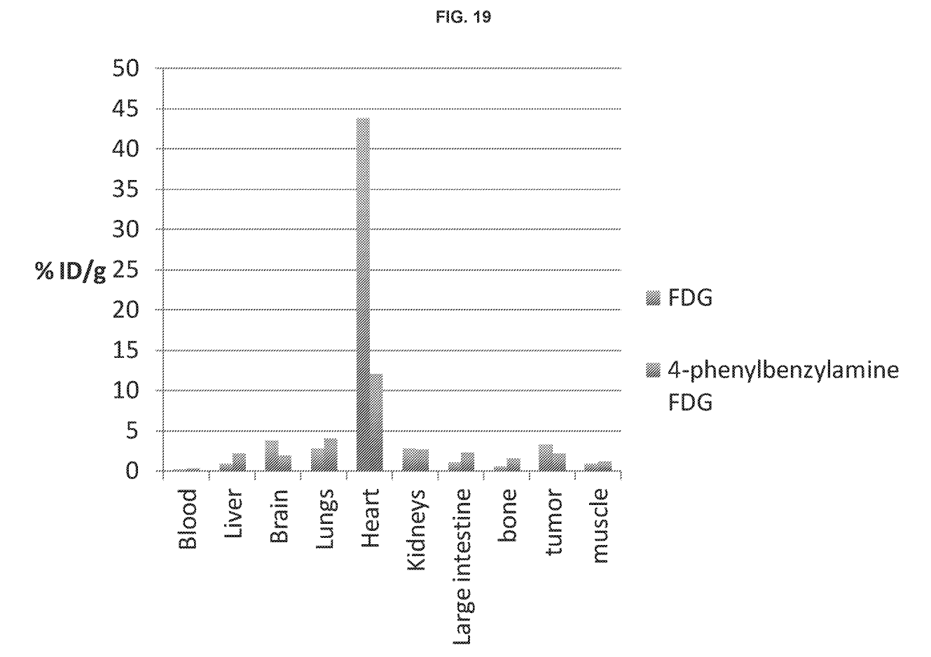

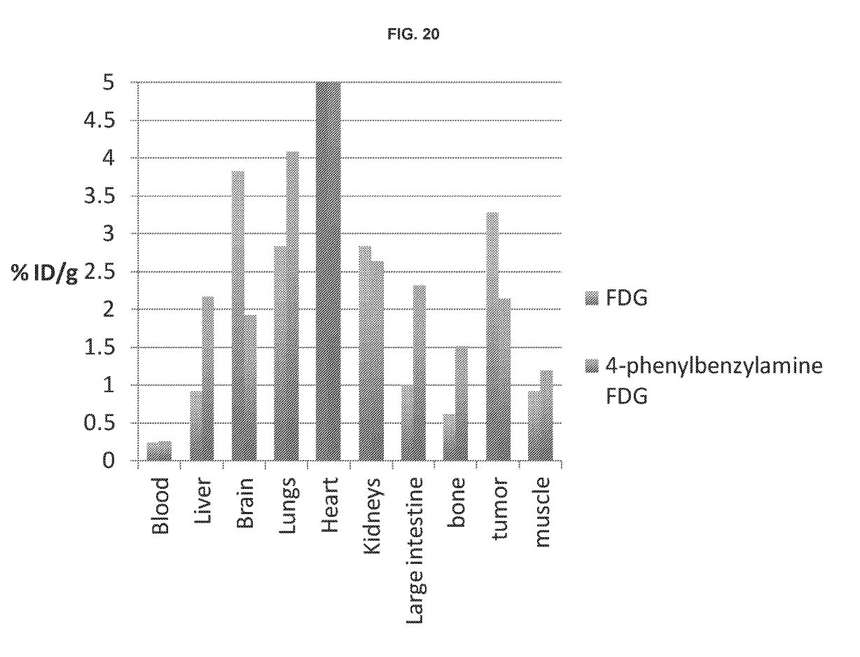

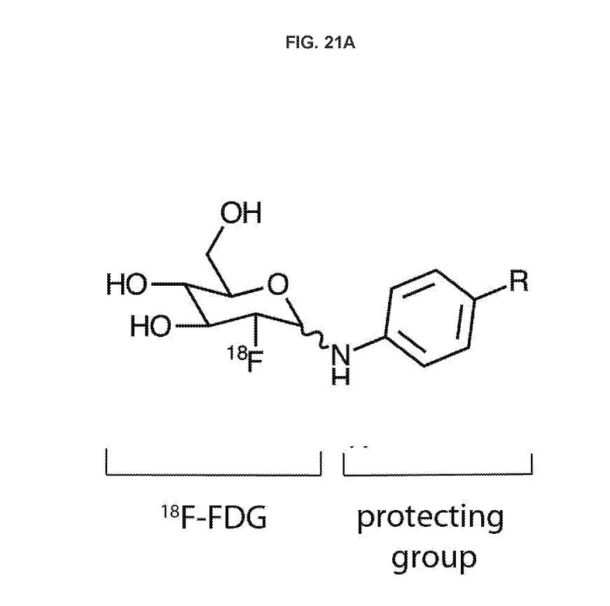

Imaging was performed using standard micro-positron emission tomography (microPET) techniques and data analysis. In brief, in a typical experiment, 100-200 μCi of FDGamine is injected into a mouse via a tail vein catheter. Following incubation time ranging from zero-one hour, the animals are imaged in a Siemens Inveon microPET-CT system. The images are reconstructed using attenuation correction from the CT portion of the exam. The resulting images are analyzed using open source Amide software (FIGS. 6, 13). For quantification of uptake in tissues, regions of interest (ROI) are drawn over tissues using the CT portion of the exam. By correcting for the known injected dose, the absolute uptake in tissues can be computed using Amide software (FIG. 14-20).

1. A reaction-based positron emission tomography probe compound having a structure according to Formula (I):
A−(B)ₙ−C (I)
in which
A is a sugar moiety;
B is a linker;
C is a caging moiety selected from substituted or unsubstituted aryl and substituted or unsubstituted heteroaryl moieties; and
n is an integer selected from 0 and 1.

2. The probe according to claim 1, wherein said sugar moiety comprises positron emitting atom selected from ¹¹C, ¹³_{N,} ¹⁵O and ¹⁸F.

3. The probe according to claim 1, wherein said caging moiety comprises a member selected from substituted or unsubstituted aryl and substituted or unsubstituted heteroaryl.

4. The probe according to claim 1, wherein said sugar moiety is a deoxysugar.

5. The probe according to claim 1, wherein said sugar moiety has the structure according to Formula (II): 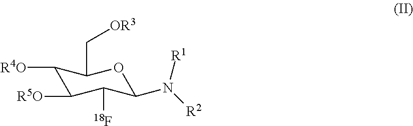
in which
R¹ and R² are independently selected from H, substituted or unsubstituted aryl substituted or unsubstituted heteroaryl, substituted or unsubstituted alkyl (e.g., benzyl), with the proviso that at least one of these radicals is other than H; and
R³-R⁵ are independently selected from H, substituted or unsubstituted alkyl, substituted or unsubstituted heteroalkyl, substituted or unsubstituted heterocycloalkyl, substituted or unsubstituted aryl and substituted or unsubstituted heteroaryl.

6. The probe according to claim 1, wherein said caging moiety has the structure according to Formula (IIIa IIIa): 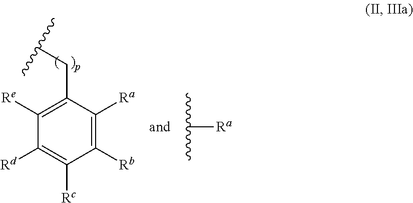
in which
R^{a}-R^{e} are members independently selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁷R⁸, —NR⁷R⁸, —OR′, —S(O)₂R⁷, —C(O)R⁷, —COOR⁷, —CONR⁷R⁸, —S(O)₂OR⁷, —OC(O)R⁷, —C(O)NR⁷R⁸, —NR⁷C(O)R⁸, —NR⁷SO₂R⁸ and —NO₂, wherein two or more of R^{a}, R^{b}, R^{d} and R^{e}, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl;
p is an integer selected from 0, 1, 2, 3,4, 5, and 6;
R⁷ and R⁸ represent members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁷ and R⁸, together with the atoms to which they are bonded, are optionally joined to form a 5- to 7-membered ring which is a member selected from substituted or unsubstituted cycloalkyl, substituted or unsubstituted heterocycloalkyl, substituted or unsubstituted aryl and substituted or unsubstituted heteroaryl.

7. The probe according to claim 1, wherein said linker has a structure according to FIG. 8.

8. A pharmaceutical formulation comprising the probe according to claim 1, and a pharmaceutically acceptable carrier.

9. A method of determining tissue uptake of the probe according to claim 1, said method comprising:
(a) administering to a subject an amount of said probe sufficient to be detected in positron emission tomography;
(b) performing said positron emission tomography on said subject, thereby acquiring a positron emission tomography data set; and
(c) determining said tissue uptake of said agent from said data set.
